(19) United States
(12) Patent Application Publication
(10) Pub. No.: US 2025/0090640 A1
(43) Pub. Date: Mar. 20, 2025

(54) METHOD OF IMPROVING THE VIABILITY OF A DENTAL IMPLANT

(71) Applicants: Bruce Mitlak, Boston, MA (US); William V. Giannobile, Boston, MA (US)

(72) Inventors: Bruce Mitlak, Boston, MA (US); William V. Giannobile, Boston, MA (US)

(21) Appl. No.: 18/778,037

(22) Filed: Jul. 19, 2024

Related U.S. Application Data
(60) Provisional application No. 63/620,195, filed on Jan. 12, 2024.
(60) Provisional application No. 63/539,445, filed on Sep. 20, 2023.

(51) Int. Cl. A61K 38/29; A61P 1/02; A61P 41/00

(52) U.S. Cl. CPC A61K 38/29; A61P 1/02; A61P 41/00

(57) ABSTRACT

Disclosed herein are methods for improving the viability of a dental implant in a subject with jawbone deterioration. The methods include treatment of the subject with abaloparatide prior to performing dental implant surgery.

### CROSS-REFERENCE TO RELATED APPLICATIONS

This application claims the benefit of U.S. Provisional Application No. 63/620,195, filed Jan. 12, 2024 and U.S. Provisional Application No. 63/539,445, filed Sep. 20, 2023, the entire contents of each of which are incorporated herein by reference in their entirety, including drawings.

### STATEMENT REGARDING FEDERALLY SPONSORED RESEARCH OR DEVELOPMENT

This invention was made with government support under DE029462 awarded by the National Institutes of Health. The government has certain rights in this invention.

### TECHNICAL FIELD

The present disclosure relates generally to improving the outcome for dental implants in patients with jawbone deterioration.

### BACKGROUND

The retention of teeth can have a profound impact on the quality of life and feelings of self-worth. Losing teeth without restoration not only impacts chewing, swallowing, and nutrition, but can impair normal speech and impacts how the patient seems themselves and how others see them. Loss of teeth without restoration is also linked to negative impacts on general health, including cardiovascular disease, diabetes, obesity, gastrointestinal disease, and cancer. Individuals with missing teeth may avoid others due to embarrassment while speaking or smiling, leading to isolation. Alternatively, denture use increases the risk of inflammation, infections, and ulcers. In addition, losing teeth without replacement causes the surrounding teeth to shift, and often leads to bacterial growth under the gum line, resulting in further loss of bone and tissue.

Restoration by implants significantly impacts overall health and mental well-being. However, loss in bone mass density can impair the viability of dental implants, and in some cases, can even prevent such a surgery due to the condition of the jaw. While medication can improve bone mass and bone density in some patients, such as when the bone loss occurs due to osteoporosis, approved medications such as bisphosphonates, romosozumab, and denosumab are associated with the risk of medication-related osteonecrosis of the jaw (MRONJ). As such, invasive dental procedures such as dental implants may be recommended prior to treatment with such agents. See e.g., Prolia® (denosumab; Amgen) injection Label, Rev. 1/2023. In some cases, treatment with such agents may be discontinued prior to invasive dental procedures. See e.g., EVENITY® (romosozumab-aqqg; Amgen) injection Label, Rev. 4/2020.

Teriparatide (Forteo®; Eli Lilly) has been previously studied in a small cohort of patients with severe, chronic periodontal disease. In this Phase I study, a 6-week course of daily teriparatide injections (20 mcg), beginning 3 days prior to periodontal surgery and concurrent with daily oral calcium (1000 mg) and vitamin D (800 IU), was reported to be associated with improved clinical outcomes relative to placebo during the year following the periodontal surgery. (See Bashutski et al., N Engl J Med; 25:2396-2405 (2010); Heath, V., Nat Rev Endocrinol 7, 4 (2011); ClinicalTrials.gov Identifier NCT00277706; Kuchler et al., Journal of Dental Research. 2011 August; 90 (8): 1001-1006). Despite these Phase I results in a trial completed well over a decade ago, teriparatide has not been further advanced in the clinic for treatment of alveolar bone loss in periodontitis.

Further, osteoanabolic agents have not been previously studied for their effects on osseointegration of dental implants, leaving patients without this potential therapeutic option. Accordingly, there exists a need for treatments which improve the viability and integrity of dental implants in order to improve the quality of life and health of the many patients who, in view of the condition of their jaw or the presence of certain conditions, are either not eligible to receive needed dental implants or are at risk of losing a dental implant.

### SUMMARY

Provided herein are methods of providing for the successful osseointegration of dental implants. In some embodiments, the method is for use in patients with a condition (e.g., jawbone deterioration, advanced age, diabetes, vascular issues) that would otherwise render implantation difficult or impossible, or patients with a high risk of poor dental implant viability (e.g., smokers, low bone mineral density, etc.). Further, individuals with osteoporosis are at a higher risk for periodontal disease and its adverse sequelae, which include alveolar bone loss and tooth loss. Accordingly, it may be desirable to increase bone formation and bone density throughout the mandible and maxilla to promote the healing of alveolar bone defects, increase primary dental implant fixation, and enhance implant osseointegration prior to or following dental implant surgery.

In one aspect is provided a method for improving the viability of a dental implant in a subject, the method comprising daily administration of an effective amount of abaloparatide to the subject for a treatment period following a dental implant surgery.

In some embodiments, the abaloparatide is administered subcutaneously.

In some embodiments, the abaloparatide is administered in an amount from about 20 meg to about 80 mcg daily.

In some embodiments, the abaloparatide is administered beginning at the time of dental implant surgery, and the daily administration is continued for a period of up to about 6 months following the dental implant surgery.

In some embodiments, the subject has previously received treatment with an anti-resorptive agent prior to the dental implant surgery.

In some embodiments, the anti-resorptive agent is a bisphosphonate.

In some embodiments, the method further comprises daily administration of an effective amount of abaloparatide to the subject for a pretreatment period prior to the dental implant surgery.

In some embodiments, the pretreatment period is up to about 3 months.

In some embodiments, the effective amount of abaloparatide administered during the pretreatment period is from about 20 mcg to about 80 mcg daily.

In some embodiments, the subject is at elevated risk for implant failure.

In some embodiments, the elevated risk comprises the presence of one or more of advanced age, diabetes, vascular disorders, low bone mineral density, or regular nicotine consumption.

In some embodiments, improving the viability comprises achieving a stable, non-painful dental implant in the subject.

In some embodiments, improving the viability comprises enhancing one or more of jawbone mass as measured by CT scan, jawbone quality as determined by histological parameters, and alveolar bone quality and volume.

In some embodiments, the dental implant surgery further comprises reconstruction for implant site development.

In some embodiments, the dental implant surgery further comprises reconstruction for peri-implantitis defects.

In some embodiments, the dental implant surgery further comprises bone grafting.

In another aspect is provided a method for improving the viability of a dental implant in a subject with jawbone deterioration, the method comprising daily administration of an effective amount of abaloparatide to the subject for a pretreatment period prior to the dental implant surgery.

In a further aspect is provided method of preparing a jaw for a dental implant surgery in a subject with jawbone deterioration, the method comprising daily administration of an effective amount of abaloparatide to the subject for a pretreatment period prior to the dental implant surgery.

In some embodiments, the jawbone deterioration is caused by at least one of: advanced periodontitis, periodontal disease, osteoporosis, a prior extraction that was not replaced, unanchored dentures or bridgework, trauma, osteomyelitis, tumors, developmental deformities, sinus deficiencies, or misalignments.

In some embodiments, the abaloparatide is administered in an amount from about 20 meg to about 80 mcg daily.

In some embodiments, the abaloparatide is administered subcutaneously.

In some embodiments, the pretreatment period is up to about 3 months before the dental implant surgery.

In some embodiments, the method further comprises continuing administration of the abaloparatide for up to about 6 months following the dental implant surgery.

In some embodiments, the subject has previously received treatment with an anti-resorptive agent prior to the dental implant surgery.

In some embodiments, the anti-resorptive agent is a bisphosphonate.

In some embodiments, improving the viability comprises achieving a stable, non-painful dental implant in the subject.

In some embodiments, improving the viability comprises enhancing one or more of jawbone mass as measured by CT scan, jawbone quality as determined by histological parameters, and alveolar bone quality and volume.

In some embodiments, preparing a jaw for a dental implant surgery comprises enhancing one or more of jawbone mass as measured by CT scan, jawbone quality as determined by histological parameters, or alveolar bone quality and volume.

In some embodiments, preparing a jaw for a dental implant surgery comprises decreasing jawbone flex from an initial jawbone flex, the jawbone flex measured by osteoprobe.

In yet another aspect is provided a method for providing alveolar ridge preservation in a subject in conjunction with dental implant surgery, the method comprising daily administration of an effective amount of abaloparatide to the subject for at least a pretreatment period prior to the dental implant surgery.

### BRIEF DESCRIPTION OF THE DRAWINGS

In order to provide an understanding of aspects of the technology, reference is made to the appended drawings, which are not necessarily drawn to scale. The drawings are exemplary only and should not be construed as limiting the technology. The disclosure described herein is illustrated by way of example and not by way of limitation in the accompanying drawings.

FIG. 1 is a schematic illustration of an animal model that mimics osteoporosis by ovariectomy according to a non-limiting embodiment of the disclosure.

FIG. 2A is a series of cartoon drawings and photographic images following ridge defect compromised implant surgery in the model of Example 1.

FIGS. 2B and 2C are photographic images of adverse healing outcomes and normal outcomes, respectively, at day 21 in the model of Example 1.

FIGS. 3A-3F are photomicrographs illustrating different percent bone fill in peri-implant (FIGS. 3A-3C) and osteonecrotic bone with periosteal proliferation (FIGS. 3D-3F) in the model of Example 1.

FIG. 4A is a graphical depiction of the effect of abaloparatide and placebo on bone mineral density in femoral shaft in the model of Example 1

FIG. 4B is a graphical depiction of the effect of abaloparatide and placebo on bone mineral density in intact maxillae in the model of Example 1.

FIG. 4C is a photomicrograph of a stained histology sample showing ridge desorption in the model of Example 1.

FIG. 5 is a schematic illustration of an animal model to study extraction socket healing and implant osseointegration according to a non-limiting embodiment of the disclosure.

FIGS. 6A-6H are a series of photographs showing the surgical protocol for implant placement in the model of Example 2.

FIG. 7A is a graphical illustration of bone volume fraction in the model of Example 2.

FIG. 7B is a graphical illustration of bone mineral density in the model of Example 2.

FIG. 7C is a series of transverse and sagittal view micro-CT scans at day 10 and day 42 for vehicle treated subjects in the model of Example 2.

FIG. 7D is a series of transverse and sagittal view micro-CT scans at day 10 and day 42 for abaloparatide treated subjects in the model of Example 2.

FIG. 8A is a series of implant micro-CT at day 21 and day 28 for vehicle and abaloparatide treated subjects in the model of Example 2.

FIG. 8B is a series of implant isosurface renderings at day 21 and day 28 for vehicle and abaloparatide treated subjects in the model of Example 2.

FIG. 8C is a graphical illustration of bone volume fraction at day 21 and day 28 for vehicle and abaloparatide treated subjects in the model of Example 2.

FIG. 8D is a graphical illustration of bone mineral density at day 21 and day 28 for vehicle and abaloparatide treated subjects in the model of Example 2.

FIG. 9A is a graphical illustration of trabecular bone mineral density over time for vehicle and abaloparatide treated animals in the model of Example 2.

FIG. 9B is an illustration of the region of interest (distal femur) in the model of Example 2.

FIG. 9C is a graphical illustration of trabecular bone mineral density over time for vehicle and abaloparatide treated animals in the model of Example 2.

FIG. 9D is an illustration of the region of interest (mid-diaphysis) in the model of Example 2.

FIG. 10A is a series of representative photomicrographs of Masson trichome-stained, decalcified sections at 2× magnification from vehicle and abaloparatide treated subjects in the model of Example 2, showing substantial regeneration of trabecular bone.

FIG. 10B is a graphical illustration of bone area fraction in the model of Example 2.

FIG. 10C is a graphical illustration of mineralized bone area in the model of Example 2.

FIG. 11A is a series of photomicrographs of representative, undecalcified, toluidine blue and basic fuchsin stained sections at 4× magnification from the VEH and ABL groups obtained at 21 days and 28 days post-implant placement in the model of Example 2.

FIG. 11B is a graphical illustration of bone area fraction in the model of Example 2.

FIG. 12 is schematic illustration of an animal model to evaluate the effect of ABL treatment on alveolar ridge healing and dimensions following tooth extraction (EXT) and on implant osseointegration according to a non-limiting embodiment of the disclosure as described in Example 3.

FIG. 13 is a graphical illustration of the surgical protocol for the study of Example 3 according to a non-limiting embodiment of the disclosure.

FIG. 14 is a photograph showing the custom drill and press fit implant in the study of Example 3 according to a non-limiting embodiment of the disclosure.

### DETAILED DESCRIPTION

Disclosed herein are methods for enhancing osseointegration of dental implants. The methods are useful in improving the viability of dental implants. While such methods may be useful in any subject receiving a dental implant, they may be particularly advantageous in subjects with certain conditions such as jawbone deterioration, advanced age, diabetes, vascular issues, low bone mineral density, or in smokers. Such subjects typically have a higher risk of poor implant viability. The methods disclosed herein generally increase the probability of a successful dental implantation by enhancing alveolar bone quality and/or mass.

As described above, teriparatide has been previously studied in association with dental surgery but has not been further advanced for treatment of relevant indications. Abaloparatide has been suggested as a therapy to protect against bone loss due to periodontitis (Li, et al., J Periodontol. February 2023; 94:244-255). Specifically, Li presents results of a study of abaloparatide in a mouse model of periodontal disease, which suggested abaloparatide may protect alveolar bone in experimental periodontitis largely by promoting bone regeneration via increased osteoanabolic activity. Despite preliminary indications of the potential utility of osteoanabolic agents for treatment of alveolar bone loss associated with periodontal disease, such agents have not advanced in the clinic, and have not been studied in association with dental implant surgery. Accordingly, provided herein are methods for enhancing osseointegration of dental implants comprising administration of abaloparatide following a dental implant surgery, prior to such surgery, or both. The methods are further described herein below.

### Definitions

With respect to the terms used in this disclosure, the following definitions are provided. This application will use the following terms as defined below unless the context of the text in which the term appears requires a different meaning.

The term “abaloparatide” as used herein refers to [Glu^{22,25}, Leu^{23,28,31}, Aib²⁹, Lys^{26,30}]hPTHrP(1-34)NH₂) (Ala-Val-Ser-Glu-His-Gln-Leu-Leu-His-Asp-Lys-Gly-Lys-Ser-Ile-Gln-Asp-Leu-Arg-Arg-Arg-Glu-Leu-Leu-Glu-Lys-Leu-Leu-Aib-Lys-Leu-His-Thr-Ala-NH₂, SEQ ID NO:1), a peptide analog of PTHrP (1-34). Each of the 34 amino acids in abaloparatide are alpha amino acids. Aib is 2-aminoisobutyric acid, also known as α-aminoisobutyric acid or dimethylglycine.

As used herein, the phrase “jawbone deterioration” or the like refers to inadequate bone quality and quantity for dental implant placement and viability, e.g., decreased alveolar bone mass and density of an extent sufficient to compromise the viability of a dental implant.

### Improving the Viability of a Dental Implant

In one aspect is provided a method for improving the viability of a dental implant in a subject, the method comprising daily administration of an effective amount of abaloparatide to the subject for a treatment period following a dental implant surgery.

Improving viability of a dental implant may comprise one or more outcomes. In some embodiments, improving the viability comprises achieving a stable, non-painful dental implant in the subject.

In some embodiments, improving the viability comprises enhancing jawbone mass. Enhancement of jawbone mass may be evaluated by CT scan.

In some embodiments, improving the viability comprises enhancing jawbone quality. Enhancement of jawbone quality may be determined on the basis of histological parameters.

In some embodiments, improving the viability comprises enhancing alveolar bone quality and volume. Enhancement of alveolar bone quality and volume may be evaluated by CT scan.

Parameters which may be evaluated to determine enhancement of bone quality and volume include, but are not limited to, bone mineral density, bone-to-implant contact, bone volume fraction, trabecular thickness, trabecular separation, trabecular number, total cross-sectional area, cortical bone area, cortical thickness, and cortical bone fraction of alveolar bone.

Abaloparatide is generally administered systemically. In some embodiments, systemic administration is achieved by subcutaneous injection. Accordingly, in some embodiments, the method comprises subcutaneous administration of abaloparatide.

The dosage and frequency of abaloparatide administration may vary. In some embodiments, the abaloparatide is administered in an amount from about 20 meg to about 80 meg daily. In some embodiments, the abaloparatide is administered in an amount of 80 meg daily.

The administration of abaloparatide may be initiated at various times prior to or following dental implant surgery. In some embodiments, the abaloparatide is administered beginning at the time of dental implant surgery, and the daily administration is continued for a period of up to about 6 months following the dental implant surgery.

In some embodiments, the method further comprises daily administration of an effective amount of abaloparatide to the subject for a pretreatment period prior to the dental implant surgery.

In some embodiments, the pretreatment period is up to about 3 months, such as about 1 month, about 2 months, or about 3 months prior to the dental implant surgery.

The amount of abaloparatide administered during the pretreatment period may be more than, less than, or the same as the amount administered following the surgery. In some embodiments, the effective amount of abaloparatide administered during the pretreatment period is from about 20 meg to about 80 meg daily. In some embodiments, the effective amount of abaloparatide administered during the pretreatment period is 80 mcg daily.

In some embodiments, the subject is at elevated risk for implant failure. A number of preexisting conditions or risk factors may result in increased risk of implant failure, such as osteoporosis, age, diabetes, vascular disorders, low bone mineral density, cigarette smoking or use of oral nicotine containing products, or prior treatment with anti-resorptive agents. In some embodiments, the elevated risk comprises the presence of one or more of advanced age, diabetes, vascular disorders, low bone mineral density, or regular nicotine consumption.

In some embodiments, the subject has previously received treatment with an anti-resorptive agent prior to the dental implant surgery. In some embodiments, the anti-resorptive agent is a bisphosphonate. Bisphosphonates represent a class of medications that appear to alter normal osteoclastic function and may affect angiogenesis. These drugs are utilized to decrease osteoporosis, slow the progression of Paget Disease and prevent osseous spread of neoplasms such as multiple myeloma, breast carcinoma and prostate carcinoma. Examples of bisphosphonates include, but are not limited to, risedronate, ibandronate, alendronate, etidronate, and zoledronic acid. In some embodiments, the subject has previously received treatment with a monoclonal antibody designed to prevent osteoclastic maturation (e.g., denosumab).

In some embodiments, the subject at elevated risk for implant failure has jawbone deterioration to an extent such that the viability of the dental implant is at risk. For example, such jawbone deterioration may occur as a result of osteoporosis or following therapy with bone anti-resorptive agents as described above. In certain circumstances, use of such agents may lead to osteonecrosis or a general reduction in bone quality/volume such that the bone quality and quantity is deemed inadequate for dental implant placement and viability (e.g., by virtue of decreased alveolar bone mass and density). It would be desirable to provide such subjects with the opportunity to undergo dental implant surgery with enhanced potential for a successful outcome.

Accordingly, in another aspect is provided a method for improving the viability of a dental implant in a subject with jawbone deterioration, the method comprising daily administration of an effective amount of abaloparatide to the subject for a pretreatment period prior to the dental implant surgery.

In some embodiments, the subject has one or more of advanced age, diabetes, vascular disorders, low bone mineral density. In some embodiments, the subject regularly consumes nicotine (e.g., cigarette smoking, chewing tobacco, and the like).

In some embodiments, the subject has previously received treatment with an anti-resorptive agent prior to the dental implant surgery. In some embodiments, the anti-resorptive agent is a bisphosphonate. In some embodiments, the subject has previously received treatment with a monoclonal antibody designed to prevent osteoclastic maturation (e.g., denosumab).

The pretreatment period length of time may vary. In some embodiments, the pretreatment period is up to about 3 months, such as about 1 month, about 2 months, or about 3 months prior to the dental implant surgery.

Generally, the abaloparatide is administered systemically. In some embodiments, systemic administration is achieved by subcutaneous injection. Accordingly, in some embodiments, the method comprises subcutaneous administration of abaloparatide.

The amount of abaloparatide administered during the pretreatment period may vary. In some embodiments, the effective amount of abaloparatide administered during the pretreatment period is from about 20 mcg to about 80 mg daily. In some embodiments, the effective amount of abaloparatide administered during the pretreatment period is 80 meg daily.

In some embodiments, improving the viability comprises achieving a stable, non-painful dental implant in the subject. In some embodiments, improving the viability comprises enhancing alveolar bone quality and volume. Enhancement of alveolar bone quality and volume may be evaluated by CT scan.

Parameters which may be evaluated to determine enhancement of bone quality and volume include, but are not limited to, bone mineral density, bone-to-implant contact, bone volume fraction, trabecular thickness, trabecular separation, trabecular number, total cross-sectional area, cortical bone area, cortical thickness, and cortical bone fraction of alveolar bone.

### Preparing a Jaw for a Dental Implant Surgery

In a further aspect is provided method of preparing a jaw for a dental implant surgery in a subject with jawbone deterioration, the method comprising daily administration of an effective amount of abaloparatide to the subject for a pretreatment period prior to the dental implant surgery.

In some embodiments, the jawbone deterioration is caused by at least one of: advanced periodontitis, periodontal disease, osteoporosis, a prior extraction that was not replaced, unanchored dentures or bridgework, trauma, osteomyelitis, tumors, developmental deformities, sinus deficiencies, or misalignments.

In some embodiments, the subject has one or more of advanced age, diabetes, vascular disorders, low bone mineral density. In some embodiments, the subject regularly consumes nicotine (e.g., cigarette smoking, chewing tobacco, and the like).

In some embodiments, the subject has previously received treatment with an anti-resorptive agent prior to the dental implant surgery. In some embodiments, the anti-resorptive agent is a bisphosphonate. In some embodiments, the subject has previously received treatment with a monoclonal antibody designed to prevent osteoclastic maturation (e.g., denosumab).

The pretreatment period length of time may vary. In some embodiments, the pretreatment period is up to about 3 months, such as about 1 month, about 2 months, or about 3 months prior to the dental implant surgery. In some embodiments, the pretreatment period is up to about 3 months before the dental implant surgery.

Generally, the abaloparatide is administered systemically. In some embodiments, systemic administration is achieved by subcutaneous injection. Accordingly, in some embodiments, the method comprises subcutaneous administration of abaloparatide.

The amount of abaloparatide administered during the pretreatment period may vary. In some embodiments, the effective amount of abaloparatide administered during the pretreatment period is from about 20 mcg to about 80 mcg daily. In some embodiments, the effective amount of abaloparatide administered during the pretreatment period is 80 meg daily.

In some embodiments, the method further comprises continuing administration of the abaloparatide for up to about 6 months following the dental implant surgery, such as about 1, about 2, about 3, about 4, about 5, or about 6 months following the surgery.

In some embodiments, improving the viability comprises achieving a stable, non-painful dental implant in the subject. In some embodiments, improving the viability comprises achieving a stable, non-painful dental implant in the subject. In some embodiments, improving the viability comprises enhancing alveolar bone quality and volume. Enhancement of alveolar bone quality and volume may be evaluated by CT scan.

In some embodiments, preparing a jaw for a dental implant surgery comprises enhancing one or more of jawbone mass as measured by CT scan, jawbone quality as determined by histological parameters, or alveolar bone quality and volume. Parameters which may be evaluated to determine enhancement of bone quality and volume include, but are not limited to, bone mineral density, bone-to-implant contact, bone volume fraction, trabecular thickness, trabecular separation, trabecular number, total cross-sectional area, cortical bone area, cortical thickness, and cortical bone fraction of alveolar bone. In some embodiments, preparing a jaw for a dental implant surgery comprises decreasing jawbone flex from an initial jawbone flex, the jawbone flex measured by osteoprobe.

In some embodiments, the methods disclosed herein improve the quality and/or volume of regenerated alveolar bone in association with tooth extraction socket healing, implant osseointegration, peri-implant regeneration, or combinations thereof.

In some embodiments, the methods disclosed herein provide increased bone volume fraction (BVF), increased bone mineral density (BMD) or both at an implant site, such as in a regenerated extraction socket.

### EXEMPLIFICATION

The present invention may be further illustrated by the following non-limiting examples describing the methods. Example 1 provides an early study in a 6-month-old ovariectomized (OVX) rat model. The results of this study demonstrated that abaloparatide provided an increase in BMD in the jaw, but complications due to this older rat model, such as difficulty with implant surgery and impaired healing unrelated to abaloparatide treatment, prevented an understanding of how abaloparatide impacted the viability of the implants. As a result, Example 2 provides a proposed study in 5-week-old rats to be conducted to better understand the impact of age and abaloparatide treatment. Example 3 provides a proposed study in aged, ovariectomized female rats to evaluate abaloparatide effects on osseointegration and alveolar ridge healing and dimensions following tooth extraction (EXT).

### Example 1: Study of ABL Treatment Followed By Implants In OVX Rats

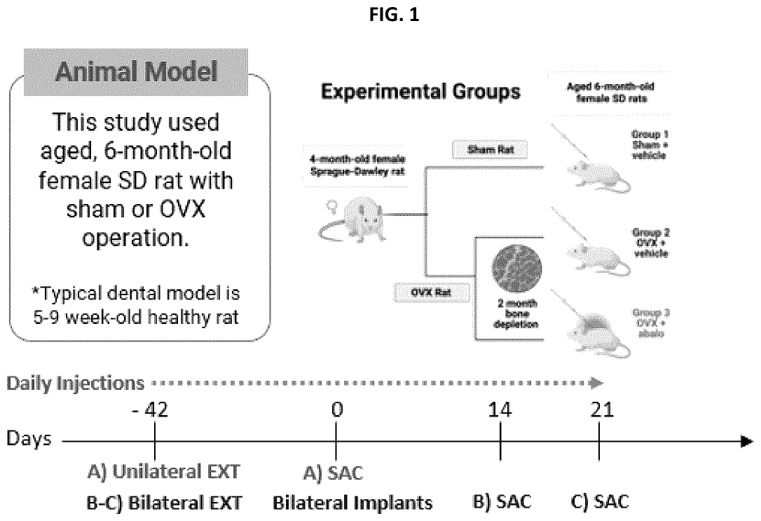

In this study, Sprague-Dawley rats were divided into three groups (Table 1) to study the impact of abaloparatide treatment on implant viability in rats in a model that mimics osteoporosis by ovariectomy (OVX). A schematic illustration of the study design is provided in FIG. 1.

TABLE 1
Study Cohorts

| | Final Sample Sizes | | |
|---|---|---|---|
| | | B) Bilateral | C) Bilateral |
| | | EXT + | EXT + |
| | Unilateral | Implants | Implants |
| Group | EXT only | (14 d) | (21 d) |
| Sham + Vehicle | n = 8 | — | — |
| OVX + Vehicle | n = 7 | n = 9, | n = 13, |
| | | Implants = 14 | Implants = 21 |
| OVX + Abalo | n = 8 | n = 7, | n = 11, |
| | | Implants = 10 | Implants = 20 |
| Total | N = 23 | N = 16 | N = 24 |
| | | Implants = 24 | Implants = 41 |

At four months, rats in the OVX groups were ovariectomized, and bone depletion was allowed to occur for two months. At this point, daily injections of abaloparatide (Abalo) or vehicle (VEH) began 42 days prior to implant surgery and were continued out to 21 days past implant surgery.

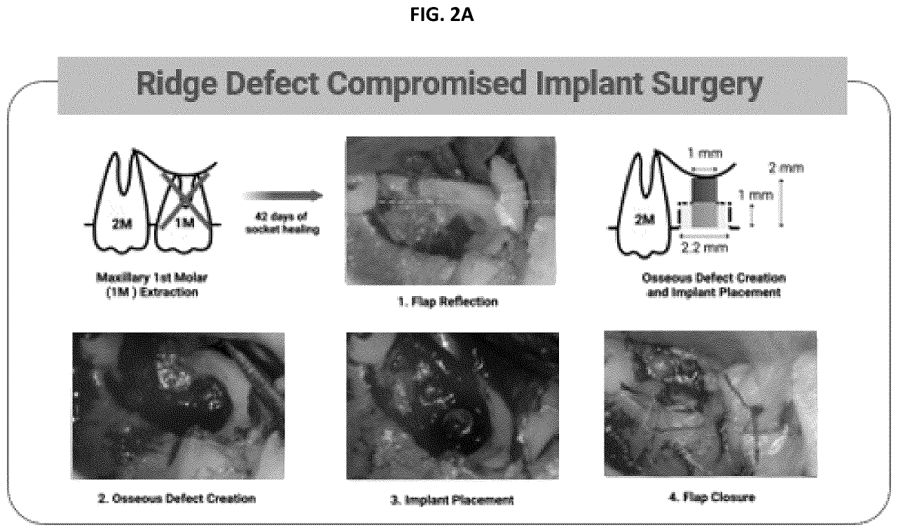

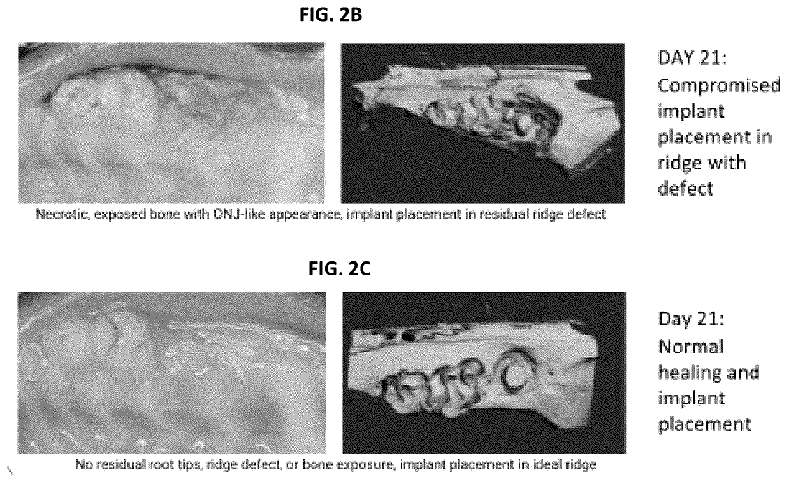

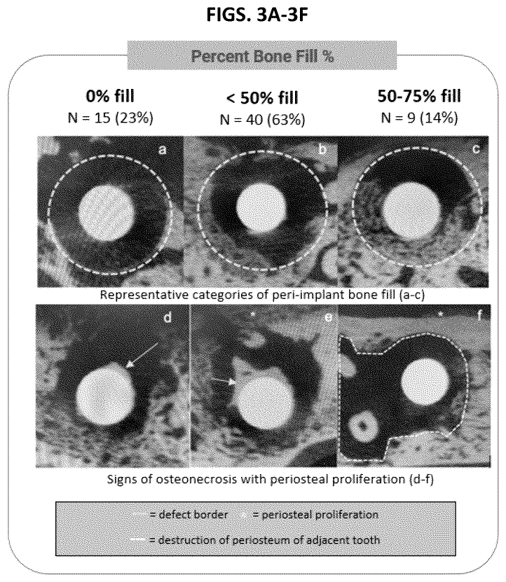

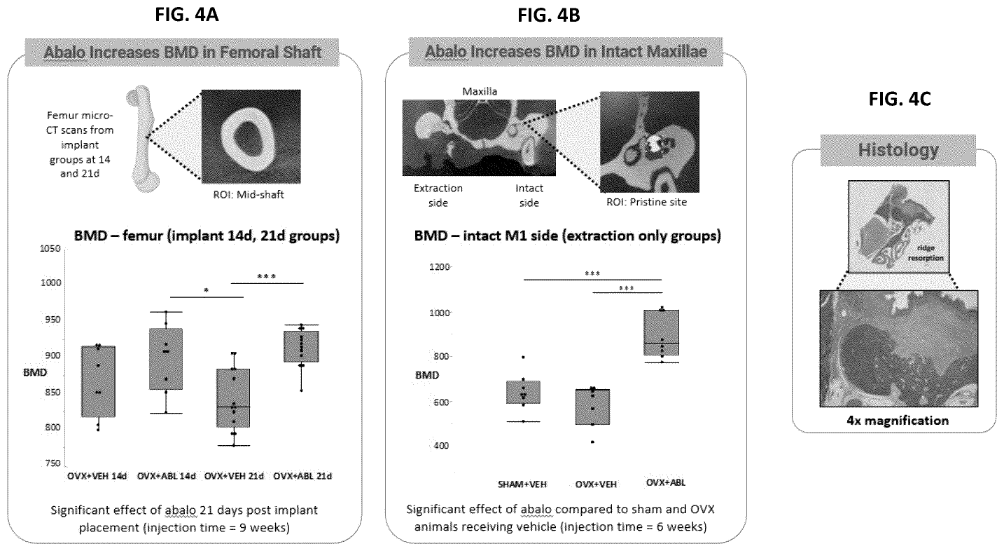

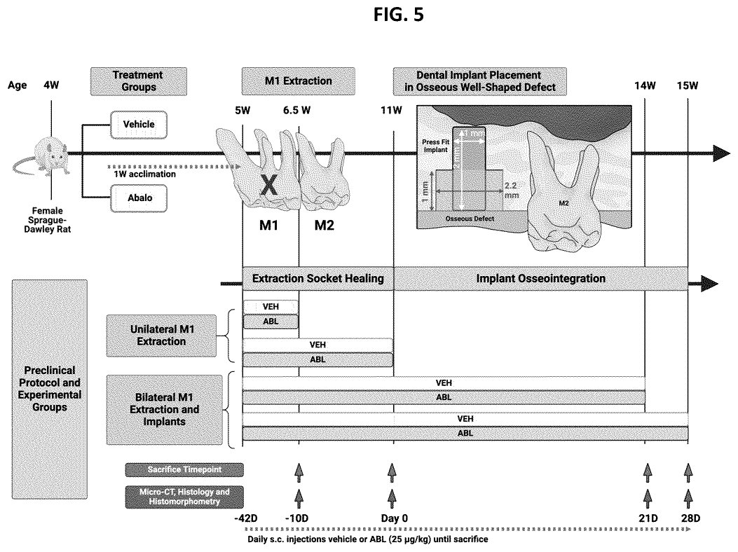

As shown in FIGS. 2A-2C, 3A-3F, and 4A-4C, altered bone metabolism in the OVX rats and residual ridge defects complicated implant placement. In addition, unexpected healing complications were observed after tooth extraction, although no relationship to abaloparatide treatment was found. However, abaloparatide did increase bone mass density in the femoral shaft and intact maxillac on the non-extraction side of the jaw (FIGS. 4A and 4B, respectively). With continued reference to FIGS. 4A and 4B, there was a 150% increase in mean BMD in OVX rats treated with ABL versus VEH.

### Example 2: Study of ABL Treatment Followed by Implants In Young Rats

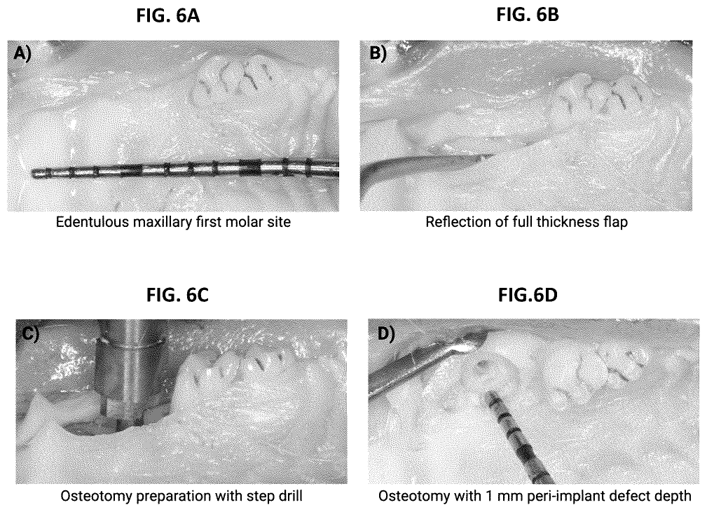

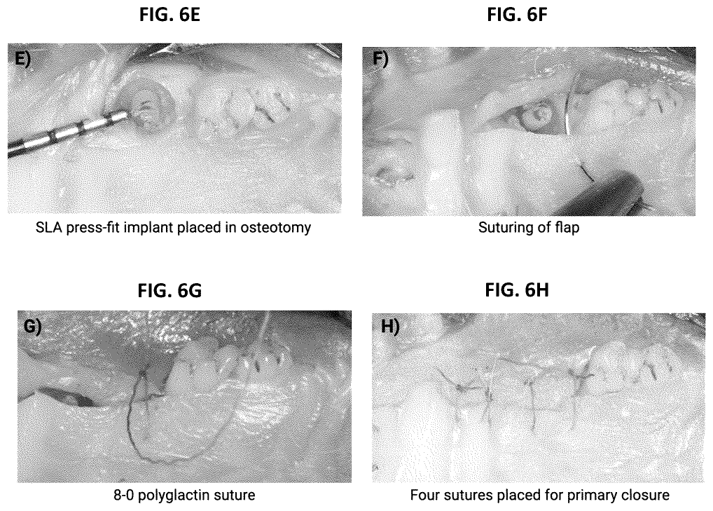

This was a study in 5-week-old rats to observe extraction socket healing and implant osscointegration. A schematic illustration of the study design is provided in FIG. 5. Healthy, 4-week-old, female Sprague-Dawley rats were housed (4 animals/cage), undergoing a 7-day acclimation period upon arrival. Thirty-two animals received unilateral extraction of the right maxillary first molar (M1); 16 animals (VEH; n=8, ABL; n=8) were sacrificed at 10 days post-extraction and 16 animals (VEH; n=8, ABL; n=8) were sacrificed at 42 days post-extraction. Thirty-nine animals received bilateral M1 extractions and were allotted 42 days of M1 socket healing prior to undergoing a second surgery for bilateral implant placement; animals were sacrificed at 21 days post-implant placement (VEH; n=8, ABL; n=9) or 28 days (VEH; n=11, ABL; n=11). Immediately following tooth extraction, daily subcutaneous injections of ABL (25 μg/kg) or vehicle (saline) were initiated until designated sacrifice timepoints. Animals in the unilateral M1 extraction group were sacrificed after 10 and 42 days of extraction socket healing. A single, trained operator performed all surgical procedures masked to the treatment allocations. Treatment allocations and surgical order were randomized by cage using a computer-generated sequence. Animals received surgical extraction of the maxillary first molar (M1) unilaterally or bilaterally using an atraumatic technique as previously described (Dunn et al., 2005 Mol Ther, 11 (2), 294-299). The surgical protocol for implant placement was also performed as previously described (Dunn et al., 2005) and is demonstrated in FIGS. 6A-6H. With reference to FIGS. 6A-6H: FIG. 6A shows surgical protocol for implant placement 6 weeks post-extraction; FIG. 6B shows a one cm crestal incision was made in the healed, edentulous M1 sites, and a full-thickness mucoperiosteal flap was elevated; FIG. 6C shows an osteotomy was performed with a custom carbide step drill under copious irrigation with sterile saline; FIG. 6D shows an osteotomy with a standardized, well-shaped osseous defect measuring 1 mm in depth in the coronal half of the osteotomy; FIG. 6E shows a 2.2 mm diameter was prepared. After bilateral surgical site preparation, press-fitting of sterile, commercially pure, solid-cylinder titanium implants (1 mm diameter, 2 mm length) with a titanium sandblasted, large grit, acid-etched surface was performed. FIG. 6F shows that surgical sites were sutured with 8-0 absorbable polyglactin 910 (Flysorb, Butterfly Italia, S.r.l, Cavenago di Brianza MB, Italy) sutures (FIG. 6G) to obtain tension-free, primary closure (FIG. 6H).

Implants healed with a submerged protocol beneath the mucoperiosteum unloaded until sacrifice. Following the extraction and implant procedures, multimodal pain control was performed with buprenorphine (1 mg/kg, s.c.) and carprofen (5 mg/kg, s.c.) immediately post-operatively, as well as with additional carprofen administration (5 mg/kg, s.c.) for 2 d. A soft diet (DictGel 76A, Clear H₂O, Westbrook, ME, and water-softened chow pellets), nutritional supplement (DictGel Recovery, Clear H₂O, Westbrook, ME), and drinking water with ampicillin (268 μg/ml) was used for 7 days post-operatively.

All animals were euthanized by carbon dioxide inhalation overdose followed by cervical dislocation at the designated sacrifice timepoints. Posterior maxillary specimens were harvested, dissected, and fixed in 10% neutral-buffered formalin for 2 days, then transferred to 70% alcohol. Each group and time point in the extraction and implant cohorts initiated with n=8 and n=12 animals, respectively. For acquisition of the micro-CT images, all specimens were oriented along the sagittal plane in 34 mm diameter holders (μCT100 Scanco Medical, Bassersdorf, Switzerland). Scan settings were as follows: voxel size 18 μm, 90 kVp, 44 μA, 0.5 mm AL filter, and integration time 1,000 ms. Scans were reconstructed and two- and three-dimensional images were generated for all specimens. Micro-CT analysis was performed following protocols described previously (Mackawa et al., 2022, Advanced Materials Interfaces, 2200531; Wang et al., 2020, J Dent Res, 99 (8), 930-937). In the extraction groups, the mesial root socket was annotated as the region of interest (ROI) and measured (length: 70 slices, threshold: HU 184). In the implant groups, the halo effect was titrated and the osseous well-shaped defect region was annotated. All annotations were performed in a masked fashion by three trained and calibrated examiners and required unanimous consensus to finalize the analysis. The newly formed bone volume fraction (BVF) and bone mineral density (BMD) were calculated for the extraction socket and peri-implant ROIs. Femur samples were assessed using the femur midshaft as the ROI for cortical BVF and BMD (length: center 100 slices, threshold: HU 330) and the distal femur (1 mm away from the growth plate) as the ROI for trabecular BMD and BVF (length: 150 slices, threshold: HU 220). Masked Micro-Ct analysis was performed by trained and calibrated examiners (BF, BY) using Scanco software (Scanco Medical AG, Brüttisellen, Switzerland). Decalcified sections of extraction sockets and undecalcified sections of implant samples were prepared for histologic and histomorphometric analyses as described previously (Maekawa et al., 2022), Microscopic images were captured with a Keyence BZ-X700E microscope (Keyence Corp. of America., Itaca, IL, USA). Histologically, the regenerated bone areas in the extraction and implant samples were assessed for bone volume (BV defined as bone area/tissue area) and mineralized bone area fraction (MB defined as bone area-osteoid areas) (Dempster et al., 2013, J Bone Miner Res, 28 (1), 2-17). Bone-to-implant contact (BIC) was also assessed in implant samples within the defect area and for the entire length of the implant (Maekawa et al., 2022). Masked histologic and histomorphometric analysis was performed by a single trained and calibrated examiner.

Analysis of peri-implant micro-CT and histological data was performed by one-way analysis of variance (ANOVA) followed by Bonferroni-corrected post hoc tests. Analysis of extraction socket micro-CT and histological data as well as femur micro-CT data was performed by Student's t-test followed by Bonferroni-Dunn correction for multiple testing. Where hypothesis testing was performed, a p-value of <0.05 was considered significant. Statistical analysis was performed using Prism 10.1.0 (GraphPad Software, Boston, MA, USA). The minimum sample size to attain statistical significance of p<0.05 with 80% power was 4 animals per group based on results for peri-implant BMD from a previous study (Yu et al., 2018, Tissue Eng Part A, 24 (21-22), 1672-1679).

### Results

### Micro-CT Findings

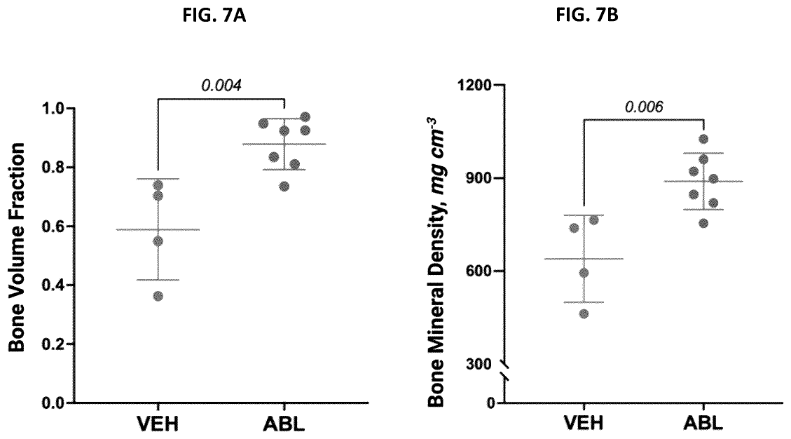

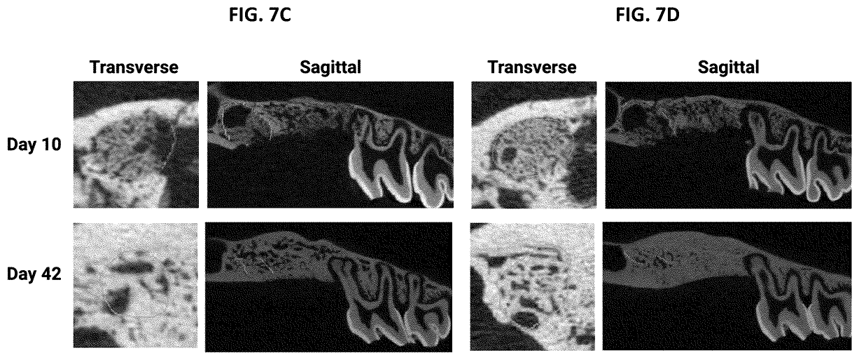

A total number of 4-7 animals per group in the unilateral extraction cohort, 5-7 animals (6-14 implants) in the 21-day implant group, and 8-10 animals (9-16 implants) in the 28-day implant group. Some specimens could not be evaluated due to implant loss or due to post-surgical complications. No statistically significant relationship to the test or control treatment in the excluded specimens was noted. Micro-CT was performed on maxillary extraction sites on 10 and 42 days after unilateral M1 extraction in rats that did not receive implants. A high degree of significant osteogenesis was observed in the VEH and ABL groups at 10 days; there were no significant differences in BVF or BMD between treatment groups after 10 days. At 42 days, ABL treatment resulted in greater bone fill and density of the regenerated bone in the extraction sockets, exhibiting statistically significant increases for BVF (p=0.004) and BMD (p=0.006) versus vehicle-treated controls (FIGS. 7A and 7B, respectively). Overall, both groups consistently demonstrated complete socket healing at 42 days; the borders between the healed socket and the surrounding native bone were nearly indistinguishable and required thresholding to detect the regenerated bone area (FIG. 7C (vehicle) and FIG. 7D (abaloparatide)).

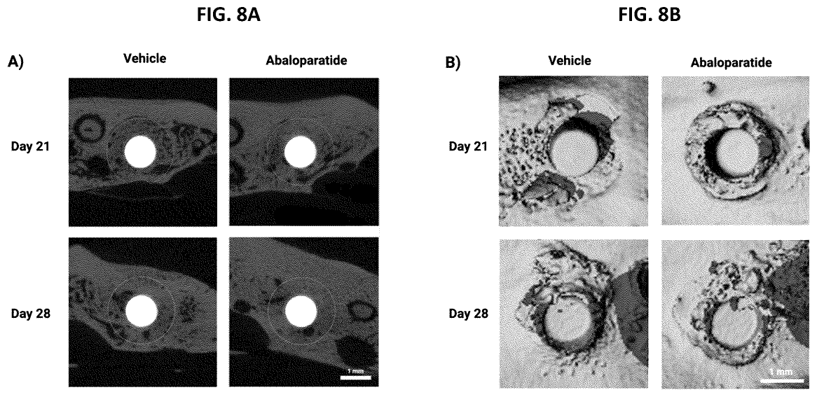

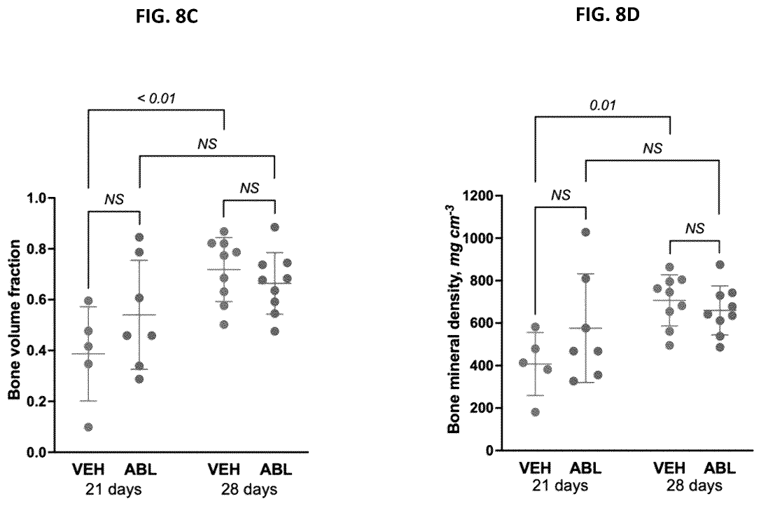

In the micro-CT analysis of the implant groups, significant bone formation was observed directly on the implant surfaces in the region of the peri-implant defects for both treatment groups at 21 d, indicating osscointegration. At 28 d, both the ABL and VEH groups exhibited high degrees of defect fill in the peri-implant region of interest. No significant differences were detected in BVF or BMD at 21 or 28 d after implant placement in the ABL-treated group compared to the group receiving VEH (FIGS. 8A and 8B; implant micro-CT and isosurface renderings, respectively). VEH-treated animals exhibited increases in BVF and BMD between the 21 day (p<0.01) and 28 day (p=0.01) time points, while ABL-treated animals did not show differences between these two time points (FIGS. 8C and 8D; BVF and BMD, respectively). These results showed that ABL accelerated bone maturation in the earlier phase of healing, reaching overall peak BVF and BMD before the VEH group by 21 days.

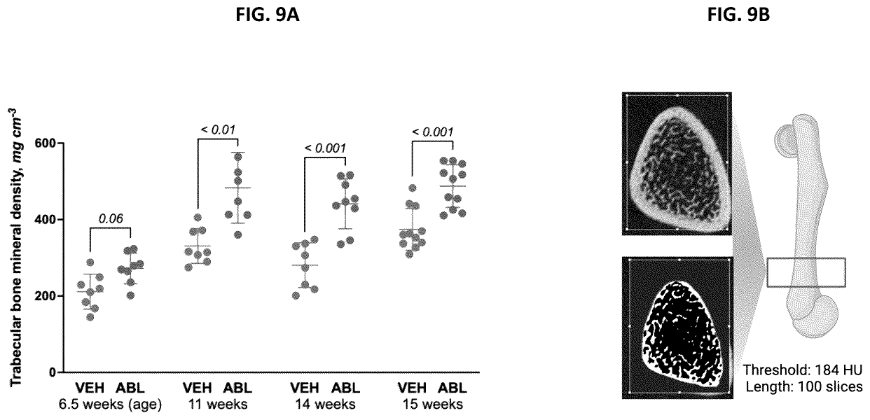

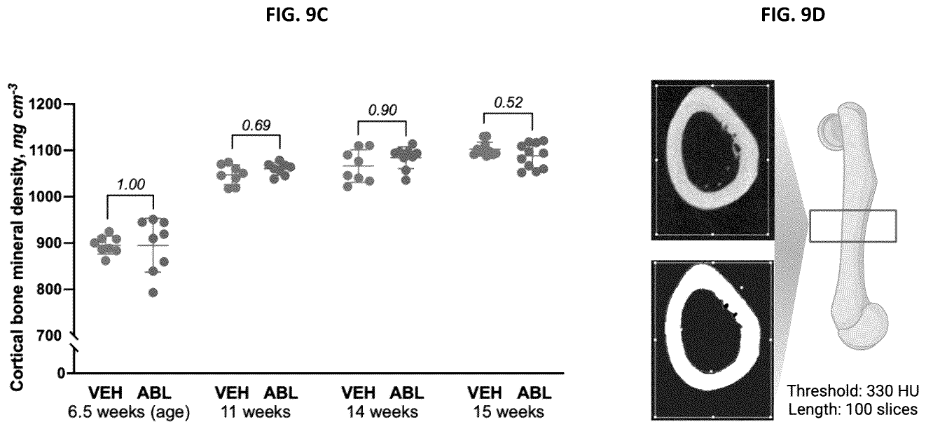

The systemic effect of abaloparatide on bone healing was confirmed through micro-CT assessment of femurs from all animals in the extraction and implant groups. Micro-CT of the distal femoral metaphysis indicated significantly greater trabecular BMD with ABL vs VEH after 6, 9, and 10 weeks of treatment (FIG. 9A). The region of interest (distal femur trabecular) is shown in FIG. 9B. No significant between-group differences were observed in the cortical BMD (FIG. 9C; region of interest (mid-diaphysis) is shown in FIG. 9D).

### Histological Findings

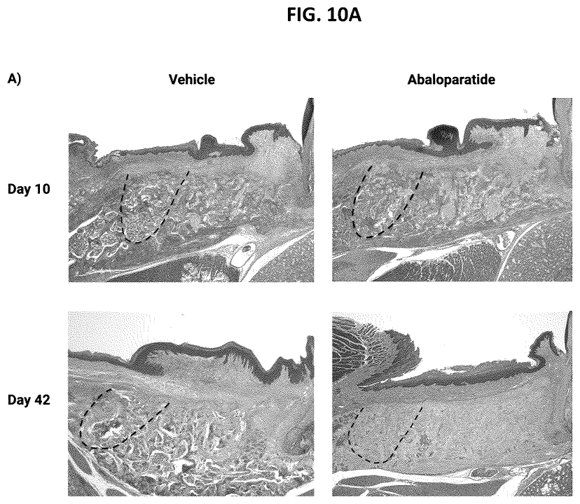

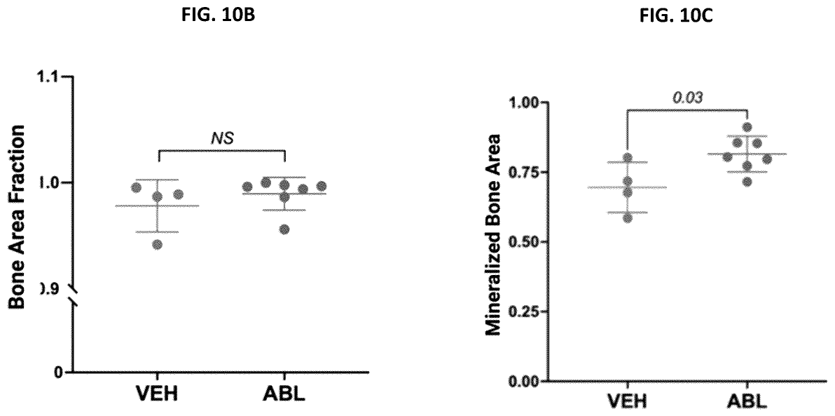

The mucosa overlying the M1 site was generally intact in all samples at 10 days and 42 days. In the 10-day groups, significant formation of woven bone extending from the borders of native bone at the socket wall was observed with a confluent appearance in most of the samples. No significant differences were detected in BV or MB at 10 days. At 42 days, both groups demonstrated over 90% BV in the sockets without significant differences in this parameter. FIG. 10A provides representative photomicrographs of Masson trichome-stained, decalcified sections at 2× magnification from each group in which the oral epithelium was intact at 10 days of healing following M1 extraction and remained intact at day 42. The ABL and VEH groups both show substantial regeneration of trabecular bone throughout the extraction sockets at 10 days. At 42 days, the ABL group example shows a denser trabecular network than the VEH control, suggesting a greater degree of bone maturation. The black dashed line delineates border of mesial root socket.

No significant differences in BV were observed at 42 days, with both groups demonstrating nearly 100% bone fill (FIG. 10B). When excluding osteoid tissue areas from the bone area, MB was significantly higher in the ABL group (p=0.03) indicating a higher degree of bone maturation with the test treatment (FIG. 10C).

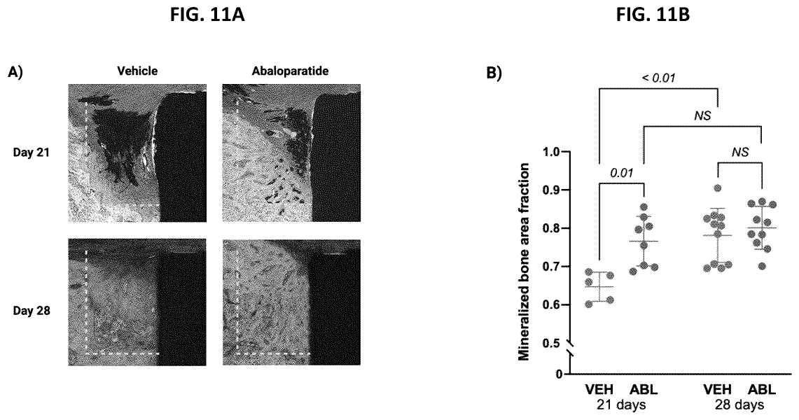

Photomicrographs of representative, undecalcified, toluidine blue and basic fuchsin stained sections at 4× magnification from the VEH and ABL groups obtained at 21 days and 28 days post-implant placement are provided in FIG. 11A. The yellow dashed line delineates the peri-implant defect border. Black areas adjacent to implants are artifacts resulting from implant particles generated during tissue sectioning. In the implant groups, MB was significantly higher at 21 days post-implant placement with ABL treatment (p=0.01) as compared to the control (FIG. 11B). A significant increase in MB was detected in the VEH group between 21 and 28 days (p=0.01) but not in the ABL group. Mean BIC in the total length of the implant surface and length of the osseous-well-shaped defect area alone were not significantly different among groups in the histomorphometric analysis. Bone to implant contact areas are provided in Table 2.

TABLE 2
Bone-to-implant contact area

| | Bone-to-implant contact; | | | Bone-to-implant contact; | | |
|---|---|---|---|---|---|---|
| Days | total surface (Mean ± SD) | | | defect area (Mean ± SD) | | |
| postop | vehicle | abaloparatide | p-value | vehicle | abaloparatide | p-value |
| 21 | 0.60 ± 0.07 | 0.59 ± 0.16 | 0.90 | 0.33 ± 0.06 | 0.42 ± 0.12 | 0.23 |
| 28 | 0.64 ± 0.15 | 0.45 ± 0.24 | 0.06 | 0.49 ± 0.20 | 0.36 ± 0.22 | 0.23 |

### Discussion

This study demonstrated that ABL treatment significantly enhanced alveolar bone regeneration in healing extraction sockets and osseous peri-implant defects. When evaluating the extraction sockets, no differences between the ABL and VEH groups were observed at the early healing timepoint of 10 days. After 42 days, the ABL group exhibited significant gains in BVF and BMD as compared to the VEH group via micro-CT assessment, as well as greater MBA in the histomorphometric analysis. In the implant groups, similar amounts of bone fill in the osseous peri-implant defects were observed between groups at both the 21-day and 28-day timepoints. At 21 days, a significantly higher amount of MBA was detected in the ABL group as compared to the VEH group, suggesting accelerated maturation of regenerated peri-implant bone in the initial phase of peri-implant healing. Further, ABL-treated animals appear to have reached peak MBA by 21 days with no difference noted between 21 and 28 days while VEH-treated animals underwent a significant increase in MBA between the two time points (p=0.002). These findings demonstrate that ABL-treated animals reached peak MBA in the defect area 1 week prior to the VEH group, in which normal healing was still ongoing. No advantage of ABL in promoting osseointegration was detected as both groups exhibited similar degrees of BIC in the defect area and along the total length of the implant. Micro-CT data from the distal femur internally validated the systemic osteogenic effect of ABL in this study and established that systemic ABL administration exerts positive anabolic effects on trabecular architecture in the long bones and jaws simultaneously. Overall, under the conditions of the present study model, VEH-treated animals in the control group demonstrated complete healing in extraction sockets, implant osseointegration, and peri-implant regeneration; however, ABL treatment further enhanced the BVF and BMD in extraction sockets and MBA in peri-implant defects.

The findings from this study suggest that ABL may be useful in clinical dental procedures requiring osseous regeneration. Particularly, the positive osteoanabolic effect of ABL show high potential for clinical utility in dental regenerative procedures to enable dental implant placements in low bone mass patients. Potential applications could include alveolar ridge preservation or reconstruction for implant site development and peri-implant bone formation in more challenging regenerative scenarios, such as immediate implant placement in extraction sockets, implant placement in ridge morphologies requiring simultaneous grafting, and reconstructive surgeries for peri-implantitis defects.

### Example 3: ABL to Treat Alveolar Bone Loss for Dental Implant Reconstruction

This study is a preclinical experiment in aged 6-month-old, female Sprague-Dawley ovariectomized (OVX) rats to evaluate the effect of ABL treatment on: 1) alveolar ridge healing and dimensions following tooth extraction (EXT), and 2) osscointegration, measured by bone-to-implant contact (BIC) and alveolar bone regeneration following implant placement. The primary objective of this study is to determine whether bone volume and quality in the OVX+ABL test group will be superior to the control groups in terms of BMD, bone volume (BV %), and bone-to-implant contact (BIC %). The aim is to compare the bone quality and volume between the test and control groups by measures of:
1) BMD, BV %, and BIC % of peri-implant alveolar bone measured by micro-CT, histomorphometry, and backscatter SEM.
2) Trabecular microarchitecture (bone volume fraction (BV/TV), trabecular thickness (Tb.Th), trabecular separation (Tb.Sp), trabecular number (Tb.N), and BMD) and cortical bone morphology (total cross-sectional area (Tt.Ar), cortical bone area (Ct.Ar), cortical thickness (Ct.Th), and cortical bone fraction (Ct.Ar/Tt.Ar)) of alveolar bone, tibiae (regions of interest=proximal metaphysis, diaphysis), femur (regions of interest=distal metaphysis, mid-diaphysis), and lumbar vertebrae as measured by micro-CT.

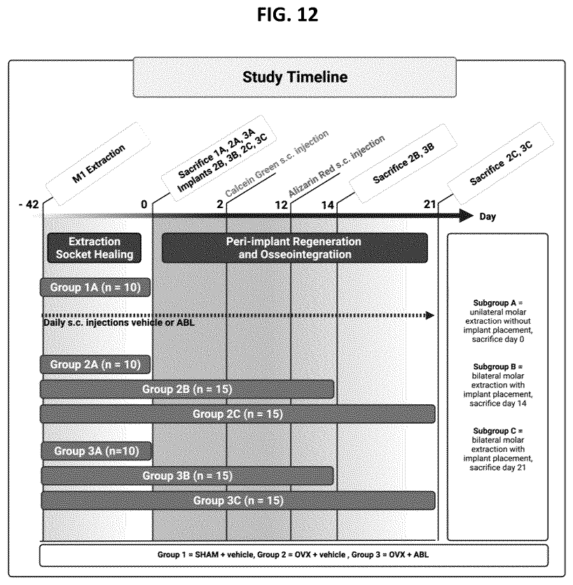

Three groups will be tested in this study. Daily subcutaneous (s.c.) injections will begin at the time of unilateral (subgroup A) or bilateral (subgroups B and C) extraction of the maxillary 1st molar (M1) and continue until sacrifice. Group 1 are sham rats receiving daily s.c. vehicle (saline) injections, Group 2 are OVX rats receiving daily s.c. vehicle (saline) injections, and Group 3 are OVX rats receiving daily s.c. ABL injections. A schematic overview of the study is provided in FIG. 12.

A total of 90 female Sprague Dawley rats will be involved in this study. At four months of age, n=80 rats will undergo ovariectomy surgery and n=10 rats will undergo sham operation. The rats will be aged for two months to allow for an adequate bone depletion period. Rats will undergo either unilateral extraction of M1 or bilateral extraction of M1 followed by bilateral osseous defect creation and implant placement.

Unilateral (Groups 1A, 2A, 3A) or bilateral (Groups 2B, 3B, 2C, 3C) extraction of M1 will occur on “Day −42” and daily injection (7 days per week) of vehicle or ABL (25 μg/kg) will continue until sacrifice timepoints. The treatment of Groups is further outlined as follows:
Group 1=Sham-operated rats receiving daily s.c. vehicle injections
   Group 1A: Unilateral M1 extraction (Day −42) and sacrifice (Day 0), (n=10)
Group 2=OVX rats receiving daily s.c. vehicle injections
   Group 2A: Unilateral M1 extraction (Day −42) and sacrifice (Day 0), (n=10)
   Group 2B: Bilateral M1 extraction (Day −42)+implant (Day 0), (n=15)
   Group 2C: Bilateral M1 extraction (Day −42)+implant (Day 0), (n=15)
Group 3=OVX rats receiving daily s.c. ABL injections
   Group 3A: Unilateral M1 extraction (Day −42) and sacrifice (Day 0), (n=10)
   Group 3B: Bilateral M1 extraction (Day −42)+implant (Day 0), (n=15)
   Group 3C: Bilateral M1 extraction (Day −42)+implant (Day 0), (n=15)

One day prior to surgery, carprofen analgesic (5 mg/kg) will be administered subcutaneously. Isoflurane will be administered to induce general anesthesia. M1 will be extracted unilaterally (Groups 1A, 2A, 3A) or bilaterally (Groups 2B, 3B, 2C, 3C) using an atraumatic technique. A sharp, curved explorer instrument will be used to luxate and extract the intact M1. If a simple extraction is not possible due to unfavorable anatomy of the tooth roots and/or bone, surgical extraction will be performed, in which the tooth crown will be sectioned into mesial and distal segments using a fissure bur under highspeed instrumentation with irrigation. The root segments then will be luxated with a spoon excavator, then removed with the explorer and cotton forceps. Immediately following extraction, the root sockets will be irrigated with 0.9% normal saline. Multimodal pain control will be performed with buprenorphine (1 mg/kg) and carprofen (5 mg/kg) immediately following surgery, as well as additional carprofen administration (5 mg/kg) 1 day post-operatively. Additionally, a soft gel diet (5% glucose/water) will be given for 1 week and drinking water with ampicillin (268 μg/ml) will be used for 2 days.

Groups 1A, 2A, and 3A (n=30) will be sacrificed after 42 days of extraction socket healing. Thirty posterior maxillary segments will be harvested (one sample per animal) and processed for micro-CT analysis of BV and BMD. Decalcified paraffin sections of the posterior maxillary segments will be prepared for histologic and histomorphometric analysis. Tibiac (regions of interest=proximal metaphysis, diaphysis), femur (regions of interest=distal metaphysis, mid-diaphysis), and lumbar vertebrae samples will be harvested from each animal and analyzed by micro-CT for their bone quality (BMD, trabecular bone microarchitecture, and cortical bone morphology). Standard micro-CT variables for trabecular bone regions and cortical bone regions will be collected.

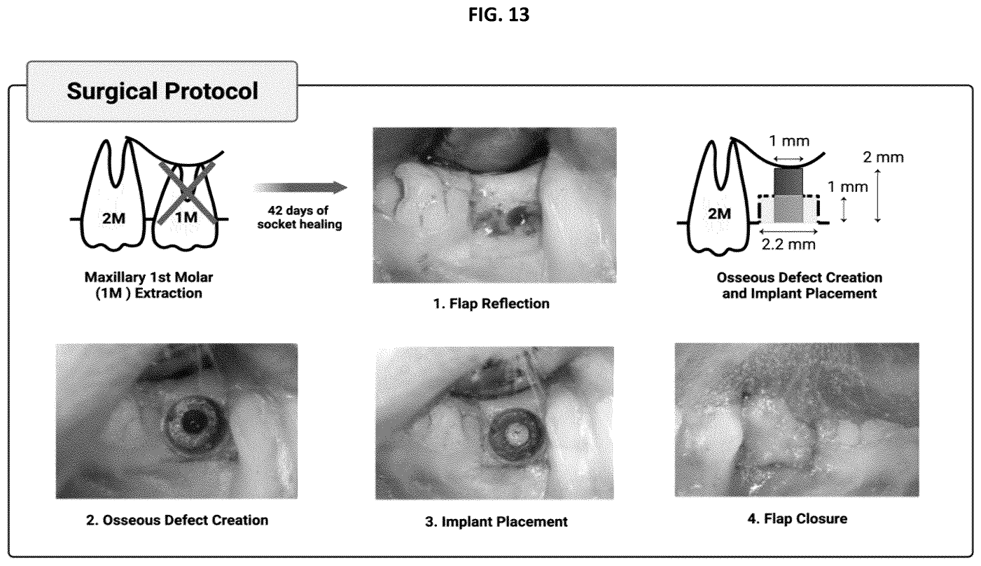

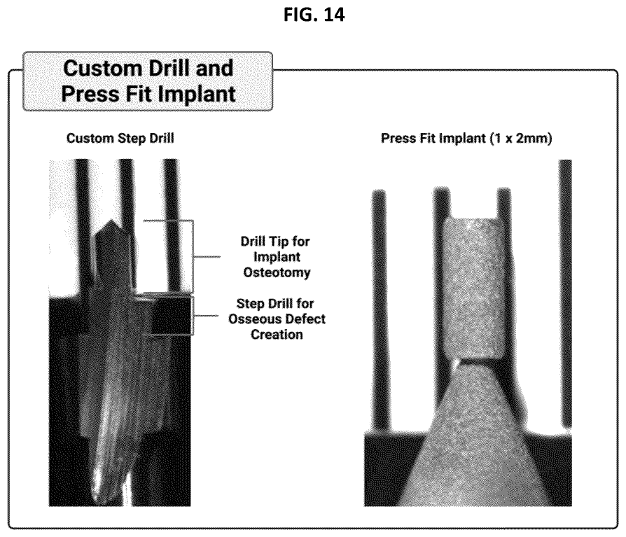

After 42 days of extraction socket healing, Groups 2B, 2C, 3B, and 3C will proceed to receive implant placement in surgically created osseous defects (FIG. 13). One day prior to surgery, carprofen analgesic (5 mg/kg) will be administered subcutaneously. Isoflurane will be administered to induce general anesthesia. A 1 cm crestal incision will be made in the edentulous ridge on both sides of the posterior maxilla. A full-thickness mucoperiosteal flap will be elevated and an osteotomy with an osseous defect component measuring 2.2 mm in diameter will be created bilaterally in the healed, left and right M1 extraction sites, with rotary instrumentation at 500 rpm under sterile saline irrigation using a custom carbide step drill. Surgical site preparation will be followed by bilateral placement of custom fabricated, sterile, commercially pure, titanium, press-fit, 1×2 mm implants (FIG. 14). The coronal half of the implant will remain exposed 1 mm above the floor of the 2.2 mm diameter osseous defect. The flap will be repositioned and closed with tissue adhesive (peri-acryl) or 8-0 absorbable polyglactin 910 (Vicryl®) sutures when indicated. The implant will remain unloaded during the healing period. Any intraoperative complications regarding the surgical procedure or primary stability will be noted.

Multimodal pain control will be performed with buprenorphine (1 mg/kg) and carprofen (5 mg/kg) immediately following surgery, as well as additional carprofen administration (5 mg/kg) 1 day post-operatively. Additionally, a soft gel diet (5% glucose/water) will be given for 1 week and drinking water with ampicillin (268 μg/ml) will be used for 2 days. The treatment of Groups is further outlined as follows:
Group 2=OVX rats receiving daily s.c. vehicle injections
   Group 2B: Sacrifice at 14 days post-implant surgery (n=15)
   Group 2C: Sacrifice at 21 days post-implant surgery (n=15)
Group 3=OVX rats receiving daily s.c. ABL injections
   Group 3B: Sacrifice at 14 days post-implant surgery (n=15)
   Group 3C: Sacrifice at 21 days post-implant surgery (n=15)

Implant placement will serve as baseline, “Day 0”, and daily vehicle or ABL injections will continue uninterrupted until scheduled sacrifice timepoints. Calcein green and alizarin red s.c. injections will be performed at days 2 and 12 respectively, to enable dynamic histomorphometric assessment of bone formation. Animals will be sacrificed in subgroups on Day 14 (Groups 2B and 3B; n=30) and Day 21 (Groups 2C and 3C; n=30). The total number of rats undergoing osseous defect creation and implant placement will be 60. 120 posterior maxillary segments with an implant placed in the M1 region will be processed for micro-CT. The micro-CT analysis will assess BMD and % bone fill with regenerated bone in the osseous defect, in addition to the standard micro-CT variables for trabecular microarchitecture and cortical morphology previously mentioned. Undecalcified polished sections will be prepared for backscatter SEM, histologic, and histomorphometric analysis of BV % and BIC %. Tibiac, femur, and vertebrae samples will be harvested from each animal and analyzed by micro-CT as well. For micro-CT, specimens will be fixed with 10% formalin for 2 days, then placed in a 34 mm diameter specimen holder and scanned using a microCT system (μCT35 Scanco Medical, Bassersdorf, Switzerland). For acquisition of the μCT images, all specimens will be oriented along the sagittal plane. Titration of the halo effect will be performed. The regenerated bone volume within the extraction sockets and peri-implant defect will be calculated by a blinded examiner. Samples will be subjected to histology and histomorphometric analysis. Samples from the extraction only groups will be decalcified in 10% EDTA, embedded in paraffin, and cut into 5 μm sections for histologic analysis. A portion of the sections with be stained with toluidine blue and basic fuchsin or hematoxylin and cosin (H&E) for histomorphometric analysis. Undecalcified polished sections will be prepared the extraction and implant groups. The samples will be dehydrated in step gradients of alcohol, infiltrated, and embedded in methyl methacrylate (MMA) using routine histological methods. Up to two cross-sectional sections of approximately 50-mm thickness will be cut along the long axis of each implant using a diamond saw at the central portion (Isomet Low Speed Saw, BUEHLER, USA). Each specimen will be attached to a plastic slide, ground down to less than 20 μm with an Ecomet 300 Pro Grinder-Polisher (Buchler, USA), and polished. After obtaining histological sections, photomicrographic images including unstained images and calcein-labeling fluorescence images will be captured. Back scattered electron (BSE) images will be taken as well. After obtaining BSE-SEM, the sections will be stained with toluidine blue and basic fuchsin. The sections will be placed in 0.1% formic acid for 5 mins, quickly rinsed with distilled water (DW), dipped into 70% ethanol for 15 mins, and stained with 1% toluidine blue for 5 mins. After rinsing with DW, sections will be dipped in 70% ethanol for 1 min, and 1% basic fuchsin for 1 min., rinsed again with DW, dehydrated in step gradients of alcohol, and dried. After obtaining stained sections, additional photomicroscopic images will be taken.

All image data will be blinded prior to histomorphometric analysis. To minimize error, examiners will calibrate intra-rater and inter-rater reliability. Quantitative analyses will be performed using Adobe Photoshop CC 2021 software (Adobe, CA, USA) for regenerated bone area measurements and Adobe Illustrator CC 2021 software (Adobe, CA, USA) for curved line measurements (i.e., implant outline in sections and BIC portion). Statistical analysis of micro-CT and histomorphometry data will be performed using Prism 8 software (GraphPad Software, CA, USA). Comparison among multiple groups will be performed with one-way analysis of variance (ANOVA) followed by Tukey.

1. A method for improving the viability of a dental implant in a subject, the method comprising daily administration of an effective amount of abaloparatide to the subject for a treatment period following a dental implant surgery.

2. The method of claim 1, wherein the abaloparatide is administered subcutaneously.

3. The method of claim 1, wherein the abaloparatide is administered in an amount from about 20 meg to about 80 meg daily.

4. The method of claim 1, wherein the abaloparatide is administered beginning at the time of dental implant surgery, and the daily administration is continued for a period of up to about 6 months following the dental implant surgery.

5. The method of claim 1, wherein the subject has previously received treatment with an anti-resorptive agent prior to the dental implant surgery.

6. The method of claim 5, wherein the anti-resorptive agent is a bisphosphonate.

7. The method of claim 1, further comprising daily administration of an effective amount of abaloparatide to the subject for a pretreatment period prior to the dental implant surgery.

8. The method of claim 7, wherein the pretreatment period is up to about 3 months.

9. The method of claim 7, wherein the effective amount of abaloparatide administered during the pretreatment period is from about 20 meg to about 80 mcg daily.

10-11. (canceled)

12. The method of claim 1, wherein improving the viability comprises one or more of achieving a stable, non-painful dental implant in the subject, enhancing jawbone mass as measured by CT scan, enhancing jawbone quality as determined by histological parameters, and enhancing alveolar bone quality and volume.

13. (canceled)

14. A method for improving the viability of a dental implant in a subject with jawbone deterioration, the method comprising daily administration of an effective amount of abaloparatide to the subject for a pretreatment period prior to the dental implant surgery.

15. A method of preparing a jaw for a dental implant surgery in a subject with jawbone deterioration, the method comprising daily administration of an effective amount of abaloparatide to the subject for a pretreatment period prior to the dental implant surgery.

16. The method of claim 15, wherein the jawbone deterioration is caused by at least one of: advanced periodontitis, periodontal disease, osteoporosis, a prior extraction that was not replaced, unanchored dentures or bridgework, trauma, osteomyelitis, tumors, developmental deformities, sinus deficiencies, or misalignments.

17. The method of claim 14, wherein the abaloparatide is administered subcutaneously in an amount from about 20 meg to about 80 meg daily.

18. (canceled)

19. The method of claim 14, wherein the pretreatment period is up to about 3 months before the dental implant surgery.

20. The method of claim 14, further comprising continuing administration of the abaloparatide for up to about 6 months following the dental implant surgery.

21. The method of claim 14, wherein the subject has previously received treatment with an anti-resorptive agent prior to the dental implant surgery.

22. The method of claim 21, wherein the anti-resorptive agent is a bisphosphonate.

23. The method of claim 14, wherein improving the viability comprises one or more of achieving a stable, non-painful dental implant in the subject, enhancing one or more of jawbone mass as measured by CT scan, jawbone quality as determined by histological parameters, and alveolar bone quality and volume.

24-26. (canceled)
